(19) United States
(12) Patent Application Publication
(10) Pub. No.: US 2024/0058082 A1
(43) Pub. Date: Feb. 22, 2024

(54) CATHETER ROBOT COMPRISING AT LEAST TWO TRANSLATION MODULES FOR LONG FLEXIBLE MEDICAL INSTRUMENT

(71) Applicant: ROBOCATH, ROUEN (FR)

(72) Inventor: Laurent DE CROUY-CHANEL, BIHOREL (FR)

(21) Appl. No.: 18/259,558

(22) PCT Filed: Dec. 22, 2021
(86) PCT No.: PCT/EP2021/087253

§ 371 Date: Jun. 27, 2023

(30) Foreign Application Priority Data
Dec. 28, 2020 (FR) 2014196

(51) Int. Cl. A61B 34/30; A61M 25/09

(52) U.S. Cl. CPC A61B 34/30; A61M 25/09; A61B 2034/301

(57) ABSTRACT

Disclosed is a catheter robot having a longitudinal axis, and including: a catheter robot support, a distal module, including an internal longitudinal translation and rotation mechanism for an external long flexible medical instrument, a proximal module, including an internal longitudinal translation and rotation mechanism for an internal long flexible medical instrument, where the distal module is intended to be arranged between the patient and the proximal module, and where the external long flexible medical instrument surrounds, on part of the longitudinal axis, the internal long flexible medical instrument, characterized in that: the proximal module is mobile in longitudinal translation relative to the support and/or relative to the distal module, the proximal module also includes an internal mechanism for rotation of the external long flexible medical instrument, synchronized to the rotation of the internal longitudinal translation and rotation mechanism for the external long flexible medical instrument of the distal module.

### FIELD OF THE INVENTION

The invention relates to a catheter robot automating the translation and rotation of a long flexible medical instrument located between two modules each comprising an internal longitudinal translation and rotation mechanism for a long flexible medical instrument.

### BACKGROUND OF THE INVENTION

According to the prior art, the catheter robot has a longitudinal axis. The catheter robot comprises a catheter robot support, a distal module, comprising an internal longitudinal translation and rotation mechanism for an external long flexible medical instrument, a proximal module, comprising an internal longitudinal translation and rotation mechanism for an internal long flexible medical instrument. The distal module is arranged between the patient and said proximal module. The external long flexible medical instrument surrounds, over at least a part of the longitudinal axis, the internal long flexible medical instrument.

The internal long flexible medical instrument is going to have to provide two types of displacement, in particular translational. Both, a generic translational displacement, simply for being able to follow the translational movement of the external long flexible medical instrument. And also, a specific translational displacement, in order to be able to respond to the command of the user wishing a specific relative movement between the external long flexible medical instrument and the internal long flexible medical instrument. The translational movement of the internal long flexible medical instrument will be the combination of the generic translational displacement and of the specific translational displacement.

The management of this translational movement of the internal long flexible medical instrument will be both fairly complex and also could require long lengths of internal long flexible medical instrument, especially if the catheter robot comprises no longer only two, but now three coaxial long flexible medical instruments, which will often be the case in practice.

### SUBJECT MATTER OF THE INVENTION

The goal of the present invention is to provide a catheter robot at least partially remedying the aforementioned disadvantages.

More specifically, the invention aims to provide a catheter robot:
which first, simplifies the complexity of the translational movement of the long flexible medical instrument located between two modules each comprising an internal longitudinal translation and rotation mechanism for a long flexible medical instrument, by creating a variable distance between the two modules;
and which next, resolves the problem of torsion of the long flexible medical instrument located between two modules each comprising an internal longitudinal translation and rotation mechanism for a long flexible medical instrument, made greater by the creation of this variable distance between the two modules.

More precisely, the invention proposes first to be able to manage a portion of this translational movement of the internal long flexible medical instrument by an overall displacement of the assembly of the proximal module and the internal long flexible medical instrument which displace together relative to the catheter robot in order to be able to vary the distance between the distal module and the proximal module.

This variable distance between the distal module bearing the external long flexible medical instrument and the proximal module bearing the internal long flexible medical instrument is going to generate a first problem of risk of buckling of the external long flexible medical instrument. But this variable distance between the distal module bearing the external long flexible medical instrument and the proximal module bearing the internal long flexible medical instrument is especially going to make a second problem of torsion of the external long flexible medical instrument between the distal module and the proximal module preponderant.

Resolving this second problem of torsion is conceivable by using a guide for the external long flexible medical instrument between the distal module and the proximal module. But then, not only would this guide first be a relatively costly element and would have a relatively complex structure for resolving this second torsion problem, then needing to be able to deform in torsion, while also saving this deformation in torsion from the external long flexible medical instrument, but also, for reasons of sanitary hygiene, because of the immediate proximity thereof with the external long flexible medical instrument, this guide would second be a consumable element discarded each time for each new patient.

The invention next proposes, for managing this second problem of torsion of the external long flexible medical instrument, made preponderant by this variable distance between the distal module bearing the external long flexible medical instrument and the proximal module bearing the internal long flexible medical instrument, of integrating in the proximal module an internal mechanism for rotation of the external long flexible medical instrument, which would be synchronized to the rotation of the internal longitudinal translation and rotation mechanism for the external long flexible medical instrument already existing in the distal module.

This internal mechanism for rotation of the external long flexible medical instrument, incorporated in the proximal module, resolves the second preponderant problem of torsion of the external long flexible medical instrument, but it does not involve the first problem of risk of buckling of the external long flexible medical instrument generated by the creation of a variable distance between the distal module bearing the external long flexible medical instrument and the proximal module bearing the internal long flexible medical instrument.

For this purpose, the present invention proposes a catheter robot having a longitudinal axis, and comprising: a catheter robot support, a distal module, comprising an internal longitudinal translation and rotation mechanism for an external long flexible medical instrument, a proximal module, comprising an internal longitudinal translation and rotation mechanism for an internal long flexible medical instrument, where said distal module is intended to be arranged between the patient and said proximal module, and where said external long flexible medical instrument surrounds, on at least one part of the longitudinal axis, said internal long flexible medical instrument, characterized in that: said proximal module is mobile in longitudinal translation relative to the support and/or relative to said distal module, said proximal module also comprises an internal mechanism for rotation of said external long flexible medical instrument, synchronized to the rotation of said internal longitudinal translation and rotation mechanism for said external long flexible medical instrument of said distal module.

Preferably, said distal module is fixed relative to the support, said external long flexible medical instrument is a guide catheter and said internal long flexible medical instrument is a catheter. In that way the torsion problem of the guide catheter is resolved.

Preferably, said distal module is mobile in longitudinal translation relative to the support, said proximal module is mobile in longitudinal translation relative to the support, said external long flexible medical instrument is a catheter, and said internal long flexible medical instrument is a catheter guidewire. In that way the torsion problem of the catheter is resolved.

For this purpose, the present invention also proposes a catheter robot having a longitudinal axis, and comprising: a catheter robot support, a first module, comprising an internal longitudinal translation and rotation mechanism for a guide catheter, fixed relative to the support, a second module, comprising an internal longitudinal translation and rotation mechanism for a catheter, a third module, comprising an internal longitudinal translation and rotation mechanism for a catheter guidewire, characterized in that: said second module is mobile in longitudinal translation relative to the support, said third module is mobile in longitudinal translation relative the support and relative to the second module, said second module also comprises an internal mechanism for rotation of said guide catheter, synchronized on the rotation of said internal longitudinal translation and rotation module of said guide catheter of said first module, said third module, also comprising an internal mechanism for rotation of said catheter, synchronized with the rotation of said internal longitudinal translation and rotational mechanism for the catheter of said second module.

According to embodiments of the invention, in order to be able to use OTW (Over the Wire) type catheters, the drive members which control the movements of the guide catheter, of the catheter, and of the catheter guidewire are going to see their parts being separated from each other in order to allow a relative translational movement between these different drive members parts. Then, because of the spacing between the various drive member parts, the guide catheter and the catheter are going to experience a heightened risk of deformation near this spacing.

In order to resolve this type of deformation problem, a guide could be arranged between drive member parts in order to guide the displacement of the medical instruments in the space between said drive member parts. However, when the drive member parts are translationally mobile in order to allow a relative movement between them, the guide located between them is then going to have to accept the movements of said drive member parts, which is then going to require the use of a guide with a complex structure (for example telescopic or else accordion). However, for reasons of hygiene, this guide is a single-use element which is replaced with each use of the robot for each new patient.

But, in fact, the most bothersome deformation and the most complex to manage is the torsion of the medical instrument around its own axis. The deformable guide system following the movements of the drive member parts, in addition to being complex and costly because it is single-use, may also be insufficient for blocking torsion of the catheter or the guide catheter. According to preferred embodiments, the invention comprises one or more of the following characteristics which may be used separately or in partial combination thereof or in full combination thereof, with one or another of the subjects of the aforementioned invention.

Preferably, said catheter robot comprises a control unit configured for commanding the longitudinal translation of the proximal module by synchronizing it with the longitudinal translation of the external long flexible medical instrument, and for controlling the longitudinal translation of the internal long flexible medical instrument by compensating the longitudinal translation of the proximal module so as to keep the internal long flexible medical instrument stationary relative to the support.

Thus, when a first long flexible medical instrument is moved in translation by a first translational module, and when a second translational module for a second long flexible medical instrument is synchronized with this translational displacement and when simultaneously this second long flexible medical instrument does not need to be displaced in translation, then this second long flexible medical instrument may be again kept in the same area, by sending it two translational movements opposed to each other and which exactly mutually compensate, since the translational movement of the second translational module in the first direction, and the translational movement of the second long flexible medical instrument relative to the second translational module in a second direction, parallel to an opposing the first direction, the two translational movements are of equal force. The synchronization of the translational displacement of the second module relative to the first medical instrument may be achieved so as to continuously maintain a set distance from the first translational module, so as to avoid a collision between the two translational modules.

Preferably, similarly, said catheter robot comprises a control unit configured for commanding the longitudinal translation of the second module by synchronizing it with the longitudinal translation of the guide catheter, and for controlling the longitudinal translation of the catheter by compensating the longitudinal translation of the second module so as to keep the catheter stationary relative to the support.

Preferably, similarly, said catheter robot comprises a control unit configured for commanding the longitudinal translation of the third module by synchronizing it with the longitudinal translation of the catheter, and for controlling the longitudinal translation of the catheter guidewire by compensating the longitudinal translation of the third module so as to keep the catheter guidewire stationary relative to the support.

Preferably, said second module does not comprise any other internal mechanism capable of providing another movement of said guide catheter besides the rotation of said guide catheter.

Thus, while remaining very effective, the second module is greatly simplified.

Alternatively, said internal rotational mechanism for said guide catheter of said second module is also capable of providing the longitudinal translation of said guide catheter.

Preferably, said first module also comprises a single pinching device for said guide catheter, said second module also comprises a single pinching device for said catheter.

Thus, while remaining very effective, the second module is greatly simplified.

Preferably, said third module also comprises a single pinching device for said catheter guidewire.

Thus, while remaining very effective, the second module is greatly simplified.

Preferably, said first module also comprises an additional internal longitudinal translation and rotation mechanism for said guide catheter which can operate alternatively with said internal longitudinal translation and rotation mechanism for said guide catheter of said first module, said second module also comprises an additional internal longitudinal translation and rotation mechanism for said catheter which may operate alternatively with said internal longitudinal translation and rotation mechanism for said catheter of said second module, said third module also comprises an additional internal longitudinal translation and rotation mechanism for said catheter guidewire which may operate alternatively with said internal longitudinal translation and rotation mechanism for said catheter guidewire of said third module.

In that way, the translational displacement of various long flexible medical instruments is going to be able to be made more fluid and quicker, at the price however of some increased complexity of the assembly of the catheter robot. But this improvement in the fluidity and speed, making the translational displacement of the various long flexible medical instruments more natural for the practitioner is thus going to be able to perform more naturally for the practitioner, and therefore in the end, overall more safely as well as more effectively.

In an alternative, said second module also comprises an additional internal mechanism for longitudinal translation and rotation of said guide catheter which can function alternatively with said internal rotation mechanism for said guide catheter of said second module, where said internal rotational mechanism for said guide catheter of said second module can also provide longitudinal translation of said guide catheter; said third module also comprises an additional internal mechanism for longitudinal translation and rotation of said catheter which can function alternatively with said internal rotation mechanism for said catheter of said third module, where said internal rotational mechanism for said catheter of said third module can also provide longitudinal translation of said catheter.

Preferably, said catheter robot comprises: a first Y connector located between said internal rotation mechanism for said guide catheter of said second module; and said internal longitudinal translation and rotation mechanism for said catheter of said second module, and a second Y connector located between said internal rotation mechanism for said catheter of said third module and said internal longitudinal translation and rotation mechanism for said catheter guidewire of said third module.

In that way, other long flexible medical instruments or other products may be laterally added to the longitudinal axis of the catheter robot, by the side thereof, so as to next be translationally moved parallel to the long flexible medical instruments already in line along the longitudinal axis of the catheter robot.

Preferably, said first Y connector is fixed to said internal rotation mechanism for said guide catheter of said second module and to said internal longitudinal translation and rotation mechanism for said second module, and said second Y connector is fixed to said internal rotation mechanism for said catheter of said third module and to said internal longitudinal translation and rotation mechanism for said catheter guidewire of said third module.

In that way, the first Y connector and the second Y connector further improve the management of the torsion problem respectively of the guide catheter and the catheter.

Advantageously, said first Y connector is fixed to said internal rotation mechanism for said guide catheter of said second module via a first turning joint. Advantageously, said second Y connector is fixed to said internal rotation mechanism for said catheter of said third module via a second turning joint.

Preferably, on at least one part of said longitudinal axis, said guide catheter surrounds said catheter which itself surrounds said catheter guidewire.

In that way, the catheter robot can be used in a coaxial configuration where the long flexible medical instruments are all mutually coaxial, over at least a part of the length thereof.

Preferably, said second module and said third module are structurally identical with each other.

In that way, the overall structure of the catheter robot is simplified, without sacrificing the overall effectiveness thereof.

Preferably, one, several or all of the internal longitudinal translation and rotation mechanism for a long flexible medical instrument capable of being a guide catheter, a catheter or catheter guidewire, comprise: two keys being capable of coming together and separating for respectively tightening on or releasing said long flexible medical instrument, said two keys being capable of doing a synchronous longitudinal translation for translating said long flexible medical instrument, said two keys being capable of doing opposite transverse translation for turning said long flexible medical instrument around the longitudinal axis.

In that way, this catheter robot has a very good compromise between effectiveness and simplicity.

Preferably, said catheter robot does not comprise any deformable guide located around one or the other of the long flexible medical instruments.

In that way, the structure of the catheter robot is simpler and also use thereof is made less costly.

In one embodiment, one or more or all of the modules comprise a mobile platform sliding longitudinally in at least one rail.

In another embodiment, one or more or all of the modules comprise a longitudinally rolling cart.

According to another object, the invention relates to a catheter robot having a longitudinal axis, and comprising:
a catheter robot support;
a distal module, comprising an internal longitudinal translation and rotation module for an external long flexible medical instrument;
a proximal module, comprising an internal longitudinal translation and rotation module for an internal long flexible medical instrument;
said distal module being intended to be arranged between the patient and said proximal module;
said external long flexible medical instrument surrounding, over at least a part of the longitudinal axis, said internal long flexible medical instrument.
characterized in that:
said proximal module is mobile in longitudinal translation relative to the support and/or relative to said distal module;
at least one among the internal longitudinal translation and rotation module for the internal long flexible medical instrument and the internal longitudinal translation and rotation mechanism for the external flexible medical instrument is constituted of one part of a pair of keys capable of coming together and separating for respectively tightening on or releasing said long flexible medical instrument, said two keys capable of doing a synchronous longitudinal translation for translating said long flexible medical instrument, said two keys capable of doing opposite transverse translation for turning said long flexible medical instrument around the longitudinal axis, and second a single pinching device for the long flexible medical instrument.

The bulk can be limited with such a catheter robot while also allowing guiding two long flexible medical instruments in translation and rotation.

Other features and advantages of the invention will be apparent to the reader of the following description of a preferred embodiment of the invention, given as an example and with reference to the attached drawings.

### BRIEF DESCRIPTION OF THE DRAWINGS

FIG. 1 schematically shows an exemplary catheter robot for driving long flexible medical instruments according to a first possible embodiment variant according to the invention.

FIG. 2 schematically shows an exemplary catheter robot for driving long flexible medical instruments according to a second possible embodiment variant according to the invention.

FIG. 3 schematically shows an exemplary catheter robot for driving long flexible medical instruments according to a third possible embodiment variant according to the invention.

FIG. 4 schematically shows an exemplary catheter robot for driving long flexible medical instruments according to a fourth possible embodiment variant according to the invention.

### DETAILED DESCRIPTION OF THE INVENTION

In all the remainder of the text of the description, and for all the figures, catheter robot, medical robot and medical catheter robot will be used interchangeably. The longitudinal axis of the catheter robot 1 is the axis shared by the three coaxial long flexible medical instruments: the guide catheter 2, the catheter 3 and the catheter guidewire 4. According to a possible variant, it is possible to use more than three flexible medical instruments. In that way, for example, two catheters can be used, where the two catheters are thus parallel to the longitudinal axis of the catheter robot 1 and are inserted inside the guide catheter 2.

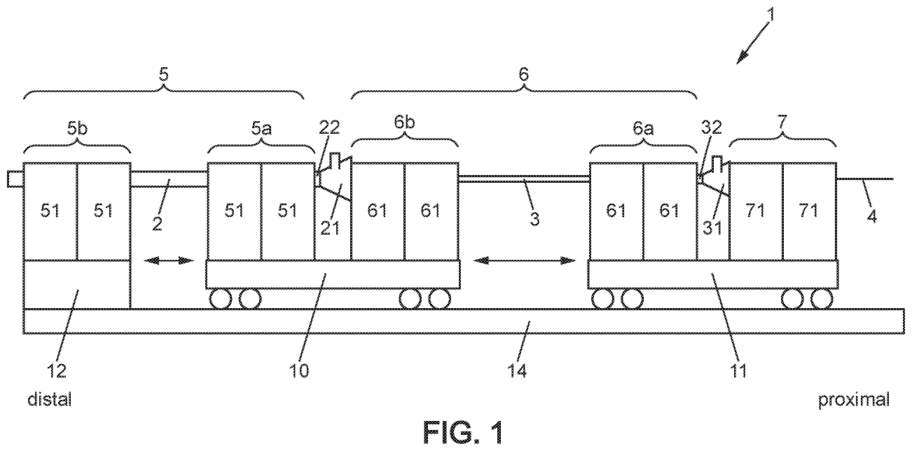

FIG. 1 schematically shows an exemplary catheter robot for driving long flexible medical instruments according to a first possible embodiment variant according to the invention.

According to a first embodiment variant of the invention, at least one drive element for long flexible medical instrument is placed at the proximal end of the guide catheter 2 and at the proximal end of catheter 3, where this drive element is configured for performing the same rotational movement at the proximal end of the long flexible medical instrument, thus blocking torsion of the long flexible medical instrument.

In the first variant shown in FIG. 1, a medical robot 1 for driving long flexible medical instruments comprises a first drive member 5 which controls the movement of a guide catheter 2, a second drive member 6 which controls the movements of a catheter 3, and a third drive member 7 which controls the movements of a catheter guidewire 4.

The catheter guidewire 4 is arranged inside the catheter 3, on which a stent or balloon may for example be mounted, where said catheter 3 is itself arranged inside the guide catheter 2.

The first drive member 5 drives the guide catheter 2 along a translational movement along the main axis of elongation, again referred to as longitudinal axis, of said guide catheter 2 and along a rotational movement around the main axis of elongation of said guide catheter 2. Similarly, the second drive member 6 drives catheter 3 along a translational movement along the main axis of elongation of said catheter 3 and along a rotational movement around the main axis of elongation of catheter 3. Similarly the third drive member 7 drives the catheter guidewire 4 along a translational movement along the main axis of elongation of said catheter guidewire 4, and along a rotational movement around the main axis of elongation of said catheter guidewire 4. The guide catheter 2 partially surrounds the catheter 3 which itself partially surrounds the catheter guidewire 4. The guide catheter 2, the catheter 3 and the catheter guidewire 4 are mutually coaxial, over a portion of their length.

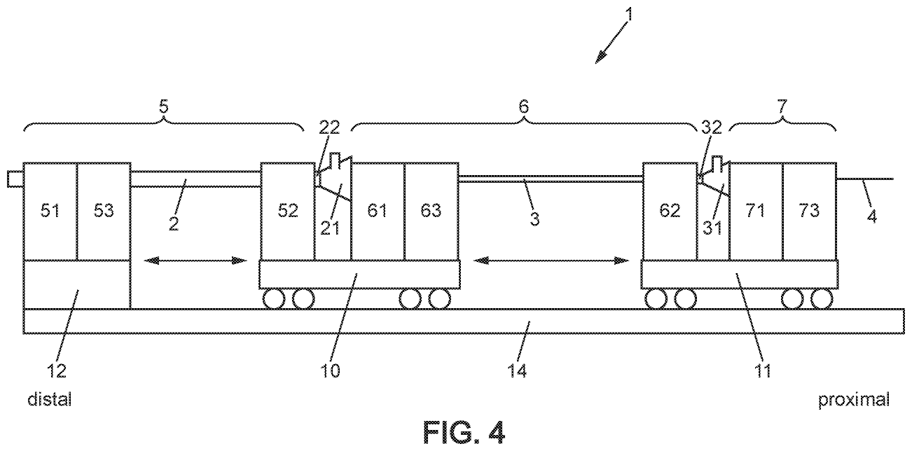

In order to control the movement of the guide catheter 2, the first drive member 5 comprises four identical elements 51 which are each configured for tightening on the guide catheter-2 and giving it a translational movement and/or a rotational movement. Each element 51 is formed by a pair of manipulator fingers, where the two fingers are located one facing the other, as is for example described in the document FR 3,044,541 (in particular see FIGS. 4a -4e and 5a-5e), incorporated here by reference. The four elements 51 are distributed into pairs separated from each other along the main axis of elongation of the guide catheter 2. A first pair 5a of elements 51 is located at the proximal end of the guide catheter 2 and is fixed to the Y connector 21 of the guide catheter 2 and more specifically to the pivot 22 of the Y connector 21. The second pair 5b of elements 51 is located at the distal end of the medical robot 1. This pair 5b of elements 51 is borne by a base 12 which is fixed relative to the support 14 (and to the case, whether it is closed, partially open or completely open) of the medical robot 1. The movement of the first pair 5a and the second pair 5b of elements 51 are synchronized such that the movement done by the first pair 5a is identical to the movement done by the second pair 5b. The fact that a single movement is imprinted by the two pairs of elements 51 serves to block deformation of the guide catheter 2 near the space between said two pairs 5a and 5b of elements 51, in particular by blocking the torsion of the guide catheter 2 around its own axis.

The first pair 5a is mobile in translation relative to the second pair 5b of elements 51 along the main axis of elongation of the guide catheter 2. In order to do this, the first pair 5a is mounted on a first mobile platform 10 which may for example be arranged on rails, and the second pair 5b of elements 51 is held fixed on the robot 1. The guide catheter 2 may thus be driven deeper or withdrawn by advancing or reversing the first mobile platform 10.

In order to control the movement of the catheter 3, the second drive member 6 comprises four identical elements 61 which are each configured for tightening on the catheter 3 and giving it a translational movement and/or a rotational movement. Each element 61 is formed by a pair of manipulator fingers, where the two fingers are located one facing the other, as is for example described in the document FR 3,044,541 (in particular see FIGS. 4a-4e and 5a-5e), incorporated here by reference. The four elements 61 are distributed into pairs separated from each other along the main axis of elongation of catheter 3. A first pair 6a of elements 61 is located at the proximal end of the catheter 3 and is fixed to the Y connector 31 of the catheter 3 and more specifically to the pivot 32 of the Y connector 31. The second pair 6b of elements 61 is located at the distal end of the medical robot 1.

The movement of the first pair 6a and the second pair 6b of elements 61 are synchronized such that the movement done by the first pair 6a is identical to the movement done by the second pair 6b. The fact that a single movement is imprinted by the two pairs of elements 61 serves to block deformation of the catheter 3 near the space between said two pairs of elements 61, in particular by blocking the torsion of the catheter 3 around its own axis.

The first pair 6a is mobile in translation relative to the second pair 6b of elements 61 along the main axis of elongation of the catheter 3. Since the second pair 6b is fixed to the Y connector 21 of the guide catheter 2, the second pair 6b of elements 61 is installed on the first mobile platform 10 in order to follow the translational movements of the guide catheter 2. Further, in order to allow driving the catheter 3 deeper or removing it, the first pair 6a of elements 61, which is fixed to the Y connector 31, is installed on a second mobile platform 11, which may for example be arranged on rails.

In order to control the movement of catheter guidewire 4, the third drive member 7 comprises two identical elements 71 which are each configured for tightening on the catheter guidewire 4 and giving it a translational movement and/or a rotational movement. Each element 71 is formed by a pair of manipulator fingers, where the two fingers are located one facing the other, as is for example described in the document FR 3,044,541 (in particular see FIGS. 4a -4e and 5a-5e). The two elements 71 are arranged in a pair which is fixed to the Y connector 31. The third drive member 7 does not require a second pair of elements 71 because the proximal end of the catheter guidewire 4 is free, the catheter guidewire 4 is not at risk of experiencing torsion or buckling. The elements 71 are arranged on the second mobile platform 11 in order to follow the translational movements of the catheter 3.

In this first variant shown in FIG. 1, the elements 51, 61 and 71 are structurally identical to each other.

The first module comprises the pair 5b of elements 51 and also the base 12. The second module comprises the first mobile platform 10, the pair 5a of elements 51, and the pair 6b of elements 61, and also the Y connector 21. The third module comprises the second mobile platform 11, the pair 6a of elements 61, and the pair 7 of elements 71, and also the Y connector 31.

The structural modules can be connected to the functional drive members. The first module comprises a part of the first drive member 5 which is located on the base 12 and also the base 12. The second module comprises the other part of the first drive member 5 which is located on the first mobile platform 10, and the part of the second drive member 6 which is located on the first mobile platform 10, and also the Y connector 21 and also the first mobile platform 10. The third module comprises the other part of the second drive member 6 located on the second mobile platform 11, and the third drive member 7 located on the second mobile platform 11, and also the Y connector 31 and also the second mobile platform 11.

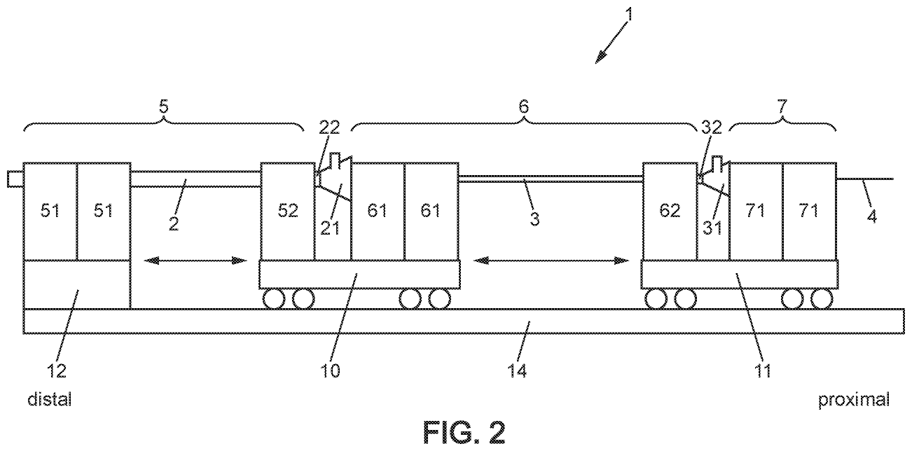

FIG. 2 schematically shows an exemplary catheter robot for driving long flexible medical instruments according to a second possible embodiment variant according to the invention.

According to a second preferred embodiment variant of the invention, in order to limit the bulk of the catheter robot, only a single drive element for long flexible medical instrument is installed at the proximal end of the guide catheter 2 and of the catheter 3.

In the second variant shown in FIG. 2, the first pair 5a of elements 51 and the first pair 6a of elements 61 from the first variant shown in FIG. 1 are each respectively replaced by a single element 52 or 62 which is configured for solely transmitting a rotational movement to the long flexible medical instrument.

Thus, in a way similar to the first variant, the first drive member 5 comprises a pair of elements 51 which are located at the distal end of the robot 1. The first drive member 5 also comprises a rotational element 52 which is fixed to the Y connector 21 of the guide catheter 2. The element 52 is an element which comprises a pair of manipulator fingers, where the two fingers are located one facing the other, as is for example described in the document FR 3,044,541. However, the element 52 is a simplified element for which the movement of the manipulator fingers along the main axis of elongation of the guide catheter 2 is eliminated; the manipulator fingers are only capable of tightening on the guide catheter 2 and giving it a rotational movement around the axis thereof. The element 52 is thus more compact than an element 51 which can do translational and rotational movements. The rotational movement given by the element 52 is synchronized with the rotational movement given by the pair of elements 51, thus blocking torsion of the guide catheter 2 around the axis thereof. During pure translational movements of the guide catheter 2, the fingers of the element 52 are tightened around the guide catheter 2 and the first mobile platform 10 displaces the element 52 in translation. During combined translational and rotational movements of the guide catheter 2, the element 52 drives the proximal end of the guide catheter 2 in rotation and said element 52 is moved in translation by the first mobile platform 10.

Similarly in the first variant, the second drive member 6 comprises a pair of elements 61 which are fixed to the Y connector 21 of the guide catheter 2 and which is installed on the first mobile platform 10. The second drive member 6 also comprises a rotational element 62 which is fixed to the Y connector 31 of the catheter 3. The element 62 is an element which comprises a pair of manipulator fingers, where the two fingers are located one facing the other, as is for example described in the document FR 3,044,541. However, the element 62 is a simplified element for which the movement of the manipulator fingers along the main axis of elongation of the catheter 3 is eliminated; the manipulator fingers are only capable of tightening on the catheter 3 and giving it a rotational movement around the axis thereof. The element 62 is thus more compact than an element 61 which can do translational and rotational movements. The rotational movement given by the element 62 is synchronized with the rotational movement given by the pair of elements 61, thus blocking torsion of the catheter 3 around the axis thereof. During pure translational movements of the catheter 3, the fingers of the element 62 are tightened around the catheter 3 and the second mobile platform 11 displaces the element 62 in translation. During combined translational and rotational movements of the catheter 3, the element 62 drives the proximal end of the catheter 3 in rotation and said element 62 is moved in translation by the second mobile platform 11.

The third drive member 7 of the second variant is identical to that of the first variant.

The first module comprises the pair of elements 51 and also the base 12. The second module comprises the first mobile platform 10, the element 52, and the pair 6b of elements 61, and also the Y connector 21. The third module comprises the second mobile platform 11, the element 62, and the pair 7 of elements 71, and also the Y connector 31.

The structural modules can be connected to the functional drive members. The first module comprises a part of the first drive member 5 which is located on the base 12 and also the base 12. The second module comprises the other part of the first drive member 5 which is located on the first mobile platform 10, and the part of the second drive member 6 which is located on the first mobile platform 10, and also the Y connector 21 and also the first mobile platform 10. The third module comprises the other part of the second drive member 6 located on the second mobile platform 11, and the third drive member 7 located on the second mobile platform 11, and also the Y connector 31 and also the second mobile platform 11.

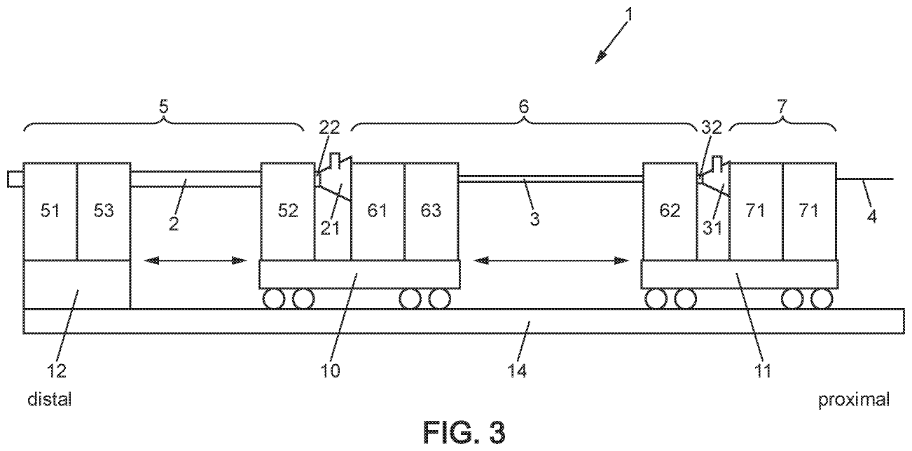

FIG. 3 schematically shows an exemplary catheter robot for driving long flexible medical instruments according to a third possible embodiment variant according to the invention.

According to a third interesting embodiment variant of the invention, in order to simplify the catheter robot, the drive element for long flexible medical instrument, or even the pair of long flexible medical drive elements might not perform a rotational drive of the long flexible medical instrument. Thus, one of the three kinematic blocks of the element may be eliminated; in fact, the kinematic block which drives the fingers for the translation of the catheter 3 may be eliminated.

The third variant shown in FIG. 3 is a variant in which the structure of the robot 1 is simplified, and in which the guide catheter 2 and the catheter 3 are no longer driven in translation and rotation with a continuous movement.

The first drive member 5 of the third variant (FIG. 3) corresponds to the first drive member of the second variant (FIG. 2) in which one of the elements 51 is replaced by a pinching device 53 which can only pinch or release the guide catheter 2 in order to keep it in position or leave it free. The pinching device 53 may for example by a pair of manipulator fingers which come to tighten or release the guide catheter 2. The function of the pinching device 53 is to hold the guide catheter 2 in position while the manipulator fingers of the elements 51 are not gripping around the guide catheter 2 during back-and-forth movements of said manipulator fingers of the element 51.

The second drive member 6 of the third variant (FIG. 3) corresponds to the second drive member 6 of the second variant (FIG. 2) in which one of the elements 61 is replaced by a pinching device 63 which can only pinch or release catheter 3 in order to keep it in position or leave it free. The pinching device 63 may for example by a pair of manipulator fingers which come to tighten or release the catheter 3. The function of the pinching device 63 is to hold the catheter 3 in position while the manipulator fingers of the elements 61 are not gripping around the catheter 3 during back-and-forth movements of said manipulator fingers of the element 61.

The first module comprises the element 51 and the element 53 and also the base 12. The second module comprises the first mobile platform 10, the element 52, and the elements 61 and 63, and also the Y connector 21. The third module comprises the second mobile platform 11, the element 62, and the pair 7 of elements 71, and also the Y connector 31.

The structural modules can be connected to the functional drive members. The first module comprises a part of the first drive member 5 which is located on the base 12 and also the base 12. The second module comprises the other part of the first drive member 5 which is located on the first mobile platform 10, and the part of the second drive member 6 which is located on the first mobile platform 10, and also the Y connector 21 and also the first mobile platform 10. The third module comprises the other part of the second drive member 6 located on the second mobile platform 11, and the third drive member 7 located on the second mobile platform 11, and also the Y connector 31 and also the second mobile platform 11.

FIG. 4 schematically shows an exemplary catheter robot for driving long flexible medical instruments according to a fourth possible embodiment variant according to the invention.

According to a fourth possible embodiment variant of the invention, which serves to further simplify the catheter robot, but however at the detriment of no longer having an entirely continuous translational movement of the long flexible medical instrument, only one rotational and translational drive element for long flexible medical instrument is installed at the distal end of the long flexible medical instrument, where this single drive element for long flexible medical instrument is coupled to a pinching device which can block any movement of the long flexible medical instrument by pinching it.

The fourth variant shown in FIG. 4 corresponds to the third variant (FIG. 3) in which the third drive member 7 also comprises an element 71 which is replaced by a pinching device 73. Such a third drive member 7 is more compact than those comprising a pair of elements 71, but the movement of the catheter guidewire 4 given by the third drive member 7 is not continuous, which makes it lose fluidity.

The first module comprises the element 51 and the element 53 and also the base 12. The second module comprises the first mobile platform 10, the element 52, and the elements 61 and 63, and also the Y connector 21. The third module comprises the second mobile platform 11, the element 62, and the elements 71 and 73, and also the Y connector 31.

The structural modules can be connected to the functional drive members. The first module comprises a part of the first drive member 5 which is located on the base 12 and also the base 12. The second module comprises the other part of the first drive member 5 which is located on the first mobile platform 10, and the part of the second drive member 6 which is located on the first mobile platform 10, and also the Y connector 21 and also the first mobile platform 10. The third module comprises the other part of the second drive member 6 located on the second mobile platform 11, and the third drive member 7 located on the second mobile platform 11, and also the Y connector 31 and also the second mobile platform 11.

Of course, the present invention is not limited to the examples and the embodiment described and shown, but there are many possible variants accessible to the person skilled in the art.

1. A catheter robot having a longitudinal axis and comprising:
a catheter robot support;
a distal module, comprising an internal longitudinal translation and rotation module for an external long flexible medical instrument;
a proximal module, comprising an internal longitudinal translation and rotation module for an internal long flexible medical instrument;
said distal module being intended to be arranged between the patient and said proximal module;
said external long flexible medical instrument surrounding, over at least a part of the longitudinal axis, said internal long flexible medical instrument.
wherein:
said proximal module is mobile in longitudinal translation relative to the support and/or relative to said distal module;
said proximal module also comprises an internal mechanism for rotation of said external long flexible medical instrument, synchronized to the rotation of said internal longitudinal translation and rotation mechanism for said external long flexible medical instrument of said distal module.

2. The catheter robot according to claim 1 wherein:
said distal module is fixed relative to the support;
said external long flexible medical instrument is a guide catheter;
said internal long flexible medical instrument is a catheter.

3. The catheter robot according to claim 1 wherein:
said distal module is mobile in longitudinal translation relative to the support;
said proximal module is mobile in longitudinal translation relative to the support;
said external long flexible medical instrument is a catheter;
said internal long flexible medical instrument is a catheter guidewire.

4. The catheter robot according to claim 1, wherein said catheter robot comprises a control unit configured for commanding the longitudinal translation of the proximal module by synchronizing it with the longitudinal translation of the external long flexible medical instrument, and for controlling the longitudinal translation of the internal long flexible medical instrument by compensating the longitudinal translation of the proximal module so as to keep the internal long flexible medical instrument stationary relative to the support.

5. A catheter robot having a longitudinal axis and comprising:
a catheter robot support;
a first module, comprising an internal longitudinal translation and rotation mechanism for a guide catheter, fixed relative to the support;
a second module, comprising an internal longitudinal translation and rotation mechanism for a catheter;
a third module, comprising an internal longitudinal translation and rotation mechanism for a catheter guidewire,
wherein:
said second module is mobile in longitudinal translation relative to the support;
said third module is mobile in longitudinal translation relative the support and relative to the second module;
said second module also comprises an internal mechanism for rotation of said guide catheter, synchronized on the rotation of said internal longitudinal translation and rotation module of said guide catheter of said first module;
said third module, also comprising an internal mechanism for rotation of said catheter, synchronized with the rotation of said internal longitudinal translation and rotational mechanism for said catheter of said second module.

6. The catheter robot according to claim 5 wherein said second module does not comprise any other internal mechanism capable of providing another movement of said guide catheter besides the rotation of said guide catheter.

7. The catheter robot according to claim 5 wherein said internal rotational mechanism for said guide catheter of said second module is also capable of providing the longitudinal translation of said guide catheter.

8. The catheter robot according to claim 5 wherein:
said first module also comprises a single pinching device for said guide catheter;
said second module also comprises a single pinching device for said catheter.

9. The catheter robot according to claim 8 wherein said third module also comprises a single pinching device for said catheter guidewire.

10. The catheter robot according to claim 5 wherein:
said first module also comprises an additional internal longitudinal translation and rotation mechanism for said guide catheter which can operate alternatively with said internal longitudinal translation and rotation mechanism for said guide catheter of said first module;
said second module also comprises an additional internal longitudinal translation and rotation mechanism for said catheter which may operate alternatively with said internal longitudinal translation and rotation mechanism for said catheter of said second module;
said third module also comprises an additional internal longitudinal translation and rotation mechanism for said catheter guidewire which may operate alternatively with said internal longitudinal translation and rotation mechanism for said catheter guidewire of said third module.

11. The catheter robot according to claim 10 wherein:
said second module also comprises an additional internal mechanism for longitudinal translation and rotation of said guide catheter which can function alternatively with said internal rotation mechanism for said guide catheter of said second module, where said internal rotational mechanism for said guide catheter of said second module can also provide longitudinal translation of said guide catheter;
said third module also comprises an additional internal mechanism for longitudinal translation and rotation of said catheter which can function alternatively with said internal rotation mechanism for said catheter of said third module, where said internal rotational mechanism for said catheter of said third module can also provide longitudinal translation of said catheter.

12. The catheter robot according to claim 5, wherein:
said catheter robot comprises:
a first Y connector located between said internal rotation mechanism for said guide catheter of said second module and said internal longitudinal translation and rotation mechanism for said catheter of second module;
a second Y connector located between said internal rotation mechanism for said catheter of said third module and said internal longitudinal translation and rotation mechanism for said catheter guidewire of said third module.

13. The catheter robot according to claim 12 wherein:
said first Y connector is fixed to said internal rotation mechanism for said guide catheter of said second module and to said internal longitudinal translation and rotation mechanism for said catheter of second module;
said second Y connector is fixed to said internal rotation mechanism for said catheter of said third module and to said internal longitudinal translation and rotation mechanism for said catheter guidewire of said third module.

14. The catheter robot according to claim 5, wherein, on at least one part of said longitudinal axis, said guide catheter surrounds said catheter which itself surrounds said catheter guidewire.

15. The catheter robot according to claim 5, wherein said second module and said third module are structurally identical with each other.

16. The catheter robot according to claim 5, wherein said catheter robot comprises a control unit configured for commanding the longitudinal translation of the second module by synchronizing it with the longitudinal translation of the guide catheter, and for controlling the longitudinal translation of the catheter by compensating the longitudinal translation of the second module so as to keep the catheter stationary relative to the support.

17. The catheter robot according to claim 5, wherein said catheter robot comprises a control unit configured for commanding the longitudinal translation of the third module by synchronizing it with the longitudinal translation of the catheter, and for controlling the longitudinal translation of the catheter guidewire by compensating the longitudinal translation of the third module so as to keep the catheter guidewire stationary relative to the support.

18. The catheter robot according to claim 1, wherein:
one, several or all of the internal longitudinal translation and rotation mechanism for a long flexible medical instrument capable of being a guide catheter, a catheter or catheter guidewire, comprise:
two keys being capable of coming together and separating for respectively tightening on or releasing said long flexible medical instrument;
said two keys being capable of doing a synchronous longitudinal translation for translating said long flexible medical instrument;
said two keys being capable of doing opposite transverse translation for turning said long flexible medical instrument around the longitudinal axis.

19. The catheter robot according to claim 1, wherein said catheter robot does not comprise any deformable guide located around one or the other of the long flexible medical instruments.

20. The catheter robot according to claim 1, wherein one or more or all of the modules comprise a mobile platform sliding longitudinally in at least one rail.

21. The catheter robot according to claim 1, wherein one or more or all of the modules comprise a longitudinally rolling cart.

22. A catheter robot having a longitudinal axis and comprising:
a catheter robot support;
a distal module, comprising an internal longitudinal translation and rotation module for an external long flexible medical instrument;
a proximal module, comprising an internal longitudinal translation and rotation module for an internal long flexible medical instrument;
said distal module being intended to be arranged between the patient and said proximal module;
said external long flexible medical instrument surrounding, over at least a part of the longitudinal axis, said internal long flexible medical instrument.
wherein:
said proximal module is mobile in longitudinal translation relative to the support and/or relative to said distal module;
at least one among the internal longitudinal translation and rotation module for the internal long flexible medical instrument and the internal longitudinal translation and rotation mechanism for the external flexible medical instrument is constituted of one part of a pair of keys capable of coming together and separating for respectively tightening on or releasing said long flexible medical instrument, said two keys capable of doing a synchronous longitudinal translation for translating said long flexible medical instrument, said two keys capable of doing opposite transverse translation for turning said long flexible medical instrument around the longitudinal axis, and second a single pinching device for the long flexible medical instrument.
